(19) United States
(12) Patent Application Publication
(10) Pub. No.: US 2011/0003308 A1
(43) Pub. Date: Jan. 6, 2011

(54) NANOPARTICLE-MEDIATED SIGNAL AMPLIFICATION

(75) Inventors: Kathy L. Rowlen, Boulder, CO (US); Amber W. Taylor, Boulder, CO (US)

Correspondence Address: HOLLAND & HART, LLP, P.O BOX 8749, DENVER, CO, 80201, US

(73) Assignee: INDEVR, INC., Boulder, CO (US)

(21) Appl. No.: 12/920,404

(22) PCT Filed: Mar. 18, 2009
(86) PCT No.: PCT/US09/37564

§ 371 Date: Aug. 31, 2010

Related U.S. Application Data
(60) Provisional application No. 61/039,515, filed on Mar. 26, 2008.

(51) Int. Cl. C12Q 1/68; C12P 19/34

(52) U.S. Cl. 435/6; 435/91.5

(57) ABSTRACT

There is described a new class or type of initiators for polymerization as a means of signal enhancement, nanoparticle initiators, and methods for amplifying signal resulting from recognition events, thereby enhancing the detection of those recognition events. Methods include amplification achieved through polymerization using a nanoparticle initiator conjugated recognition element that is not consumed during the reaction. The polymer formed as a result of the absorption of light by the nanoparticle initiator and introduction of reactive species into a surrounding polymerizable monomer solution occurs in a spatially-limited region directly surrounding the nanoparticle initiator and is indicative of the recognition event(s). In one embodiment, a semiconductor quantum dot nanoparticle initiator is utilized. In another embodiment, a metal nanoparticle is utilized. In another embodiment, the signal is detected without instrumentation. In yet another embodiment, the signal is detected via a transmission-based instrument which captures an image of the formed polymer.

### RELATED APPLICATIONS

This application claims priority to U.S. Provisional Application No. 61/039,515, filed Mar. 26, 2008, the entirety of which is hereby incorporated by reference.

### BACKGROUND

Effective global monitoring of any biological pathogen requires an inexpensive, reliable and simple analytical system that can be widely manufactured and distributed. DNA microarrays, or biochips, represent promising technology for accurate and relatively rapid pathogen identification. On a DNA microarray, the target sequence is captured by a complementary oligonucleotide (oligo) sequence that is covalently bound to a surface. The target is hybridized to a complementary label sequence that has a fluorescent moiety which can be detected by a fluorescence scanner. One of the most exciting aspects of biochip platforms is the capability to screen for multiple pathogens simultaneously.

However, several practical issues currently prevent widespread use of arrays as diagnostic tools. One example is the necessity of target amplification by polymerase chain reaction (PCR) methods when using arrays as diagnostic tools. Such PCR-based technologies are difficult to implement at point-of-care and in resource-limited settings. Although PCR is quite simple conceptually, it requires rapid thermal cycling and the hardware to facilitate such. Additionally, miniaturization and integration of existing PCR technology to a lab-on-a-chip platform is a mature field of research that still faces significant challenges. Furthermore, in its 20+ years of use, PCR has not become cost-effective for world-wide diagnostics, with the direct costs of reagents, equipment, space, personnel training, and labor reported as high as $125 USD per reaction. Reagent storage and stability are also a concern, especially in resource-limited settings where refrigeration may be intermittent or unavailable. The innate sensitivity of PCR in point-of-care settings could also be disadvantageous in terms of the potential for false positive results from cross-contamination, as tests would be performed by minimally trained personnel.

Another example of the practical issues surrounding widespread use of arrays as diagnostic tools is the need for expensive, non-field-portable microarray readers.

Still another limitation is the use of fluorescence detection in DNA microassays as described above. The use of fluorescence detections necessitates the use of expensive, high quality, non-fluorescent substrates, stringent handling conditions, and expensive and non-field-portable fluorescence microarray readers.

One manner in which some of the above described issues has been addressed is through the use of photo-polymerization for detection of bio-recognition events. The use of photo-polymerization as a means to detect bio-recognition events on surfaces in which the initiator is consumed during the reaction has been reported in, e.g., PCT/US2004/029733 and PCT/US07/61914. Signal amplification on low-density microarrays via photo-polymerization is based on a photo-active moiety, termed a photo-initiator, rather than a fluorescent moiety. Photo-initiators are generally described as a substance (other than a reactant) which, on absorption of light, generates a reactive species (ion or radical), initiates a chemical reaction or transformation, and is consumed. Once labeled with a photo-initiator, the system is covered in a solution containing “monomers” and irradiated with light of an appropriate wavelength that is absorbed by the photo-initiator. Light absorbed by the photo-initiator produces free radicals that propagate by radical addition between the surrounding monomers, thereby forming a polymer that is composed of a number of covalently bonded monomers. The photo-initiator is consumed (degraded) in the process. For a low density microarray, the process can be completed in minutes. With optimization, this system is capable of forming solid polymer located exclusively in the vicinity of the binding between the target, photo-initiator, and the substrate, thereby aiding in the identification of a bio-recognition event.

The primary drawback of this photo-polymerization method is that the photo-initiator is converted to an inactive form during the process (which is referred to herein as “consumed”). The consumption of the photo-initiator during the reaction renders it unavailable for continued excitation, which subsequently limits the sensitivity that can be achieved.

Atom transfer initiated polymerization has also been demonstrated for detection of bio-recognition events on surfaces (see, e.g., Lou et al. (2005) Anal. Chem. 77:4698-4705; Okelo and He (2007) Biosensor Bioelectron. 23:588-592). Atom transfer polymerization is based on transfer of a halogen atom from an initiator (that would be present as the label) to metal catalysts within the monomer mixture, which subsequently generate free radicals that propagate polymerization. However, as with photo-initiators, the atom transfer initiator is consumed and only one initiation event per target-label complex is achieved.

### SUMMARY

The present disclosure is directed to a new class of initiators and methods utilizing polymerization as a signal amplification mechanism. The method may comprise a non-molecular solid nanoparticle or plurality of nanoparticles conjugated to a recognition element acting as a nanoparticle initiator (NI) for the polymerization of a polymerizable solution. Significantly, the NI is not consumed during the reaction. Unique attributes of the present invention include 1) the NI is not consumed during the reaction, unlike photo-initiators and atom-transfer initiators, and 2) the polymerization reaction may proceed under ambient conditions (i.e., an inert, oxygen-free environment is not required). Based on these unique attributes, nanoparticles represent a new class of polymerization initiators that can be used to achieve signal enhancement for recognition events.

In one embodiment, a method for amplifying a recognition event between a target and a probe is disclosed. The method may comprise the steps of (a) conjugating a nanoparticle initiator to a recognition element to form a nanoparticle initiator label; (b) contacting the nanoparticle initiator label with a target element to form a nanoparticle initiator label-target complex; (c) contacting the nanoparticle initiator label-target complex with a probe element to form a nanoparticle initiator label-target-probe complex; (d) contacting the nanoparticle initiator label-target-probe complex with a polymerizable solution comprising a polymer precursor; and (e) exposing the nanoparticle initiator label-target-probe complex and the polymerizable solution to light, to form a polymer.

In another embodiment, a method for amplifying a recognition event between a target and a probe is disclosed. The method may comprise the steps of (a) contacting a target element with a probe element to form a target-probe complex; (b) conjugating a nanoparticle initiator to a recognition element to form a nanoparticle initiator label; (c) contacting the target-probe complex with the nanoparticle initiator label to form a nanoparticle initiator label-target-probe complex; (d) contacting the nanoparticle initiator label-target-probe complex with a polymerizable solution comprising a polymer precursor; and (f) exposing the nanoparticle initiator label-target-probe complex and the polymerizable solution to light, to form a polymer.

In either of the above embodiments, the method may be modified to be capable of detecting two or more target species or pathogens. In such a modification, the target element may comprise a plurality of two or more different types of target elements and the probe element may comprise a plurality of two or more different types of probe elements. Each of the plurality of two or more different types of probe elements may be immobilized at a location on a substrate and designed to form complexes with one of the two or more different types of target elements. The location on the substrate and the type of target element to which each of the plurality of the two or more different types of probe elements may be known, such that a target element may be identified based on the location on the substrate where the polymer forms.

### BRIEF DESCRIPTION OF THE DRAWINGS

FIGS. 1a-1c illustrate elements of nanoparticle initiated polymerization on an array.

FIGS. 2a-2d are schematic diagrams illustrating modes of nanoparticle initiated polymerization on an array according to embodiments disclosed herein.

FIG. 3 is a representative image of an experiment demonstrating hybridization of biotinylated oligonucleotide labeled with a streptavidin conjugated quantum dot nanoparticle initiator label to a 3×3 array of an immobilized oligonucleotides.

FIG. 4 is a micro array layout for hybridization of biotinylated Streptococcus pyogenes PCR product labeled with a streptavidin conjugated quantum dot nanoparticle initiator label to an immobilized oligonucleotide, as well as representative images resulting from genomic starting material 404 and clinical material 405.

FIG. 5 is a limit of detection comparison of nanoparticle initiated polymerization with fluorescence showing fluorescence signal as a function of target concentration.

FIG. 6 is a representative image demonstrating proof-of-concept for polymerization initiated by silver nanoparticle initiators, spotted in a 5×3 array.

FIGS. 7a-7b are schematic diagrams illustrating modes attaching a nanoparticle initiator to a target during a PCR-type amplification reaction according to embodiments disclosed herein.

### DETAILED DESCRIPTION

Exemplary embodiments illustrate methods for achieving signal amplification by a new mechanism involving nanoparticle initiated polymerization. Generally speaking, a target may be labeled with a nanoparticle initiator label. The labeled target may then be complexed with a probe due to a recognition event between the target and probe. Alternatively, a target may be complexed with a probe due to a recognition event between the target and probe, and the target-probe complex may be subsequently labeled with the nanoparticle initiator label. The label-target-probe complex may then be surrounded by a polymerizable solution. Upon exposure to light, the nanoparticle initiator may generate a reactive species that results in the formation of a polymer in the vicinity of the recognition event (i.e., where the target-probe complex formed).

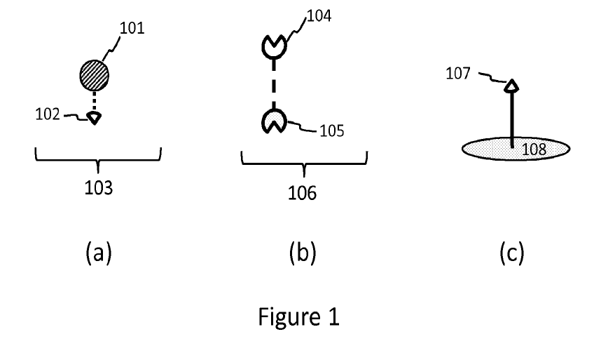

FIGS. 1a-1c illustrate three elements of the embodiments disclosed herein. In FIG. 1a, a nanoparticle initiator (NI) 101 may be conjugated to a recognition element 102, thereby forming a NI label 103. In FIG. 1b, recognition element 104 and recognition element 105 may comprise target element 106. In FIG. 1c, a probe element 107 may be immobilized on substrate 108.

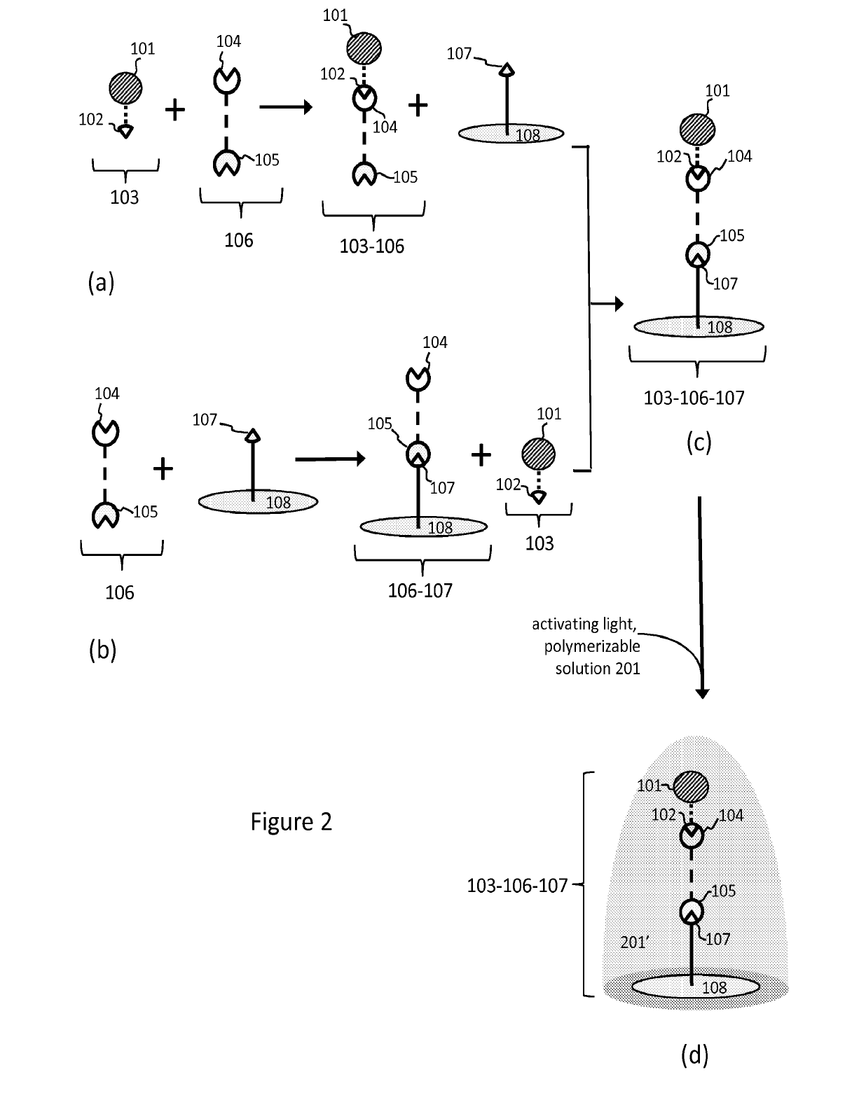

In one embodiment illustrated in FIG. 2a, NI label 103 may be contacted with target element 106, thereby forming a NI label-target complex 103-106. NI label-target complex 103-106 may then be contacted with probe element 107, thereby forming a NI label-target-probe complex 103-106-107. Probe element 107 may be immobilized on substrate 108. The formation of NI label-target-probe complex 103-106-107, and, more specifically, the interaction between recognition element 105 on target element 106 and probe element 107 that causes the two elements to bind together can be used to detect a pathogen. That is to say, probe element 107 may be designed to complex with only target element 106, and the fact that probe element 107 and target element 106 complex may signify the presence of target element 106.

In another embodiment illustrated in FIG. 2b, the method may comprise an alternate means of attaching a NI to a target-probe complex. Target element 106 may first be contacted with probe element 107, thereby forming a target-probe complex 106-107. Target-probe complex 106-107 may then be contacted with NI label 103 to form a NI label-target-probe complex 103-106-107. Probe element 107 may be immobilized on a substrate 108. The complexing between probe element 107 and target element 106 may represent the recognition event discussed in greater detail previously.

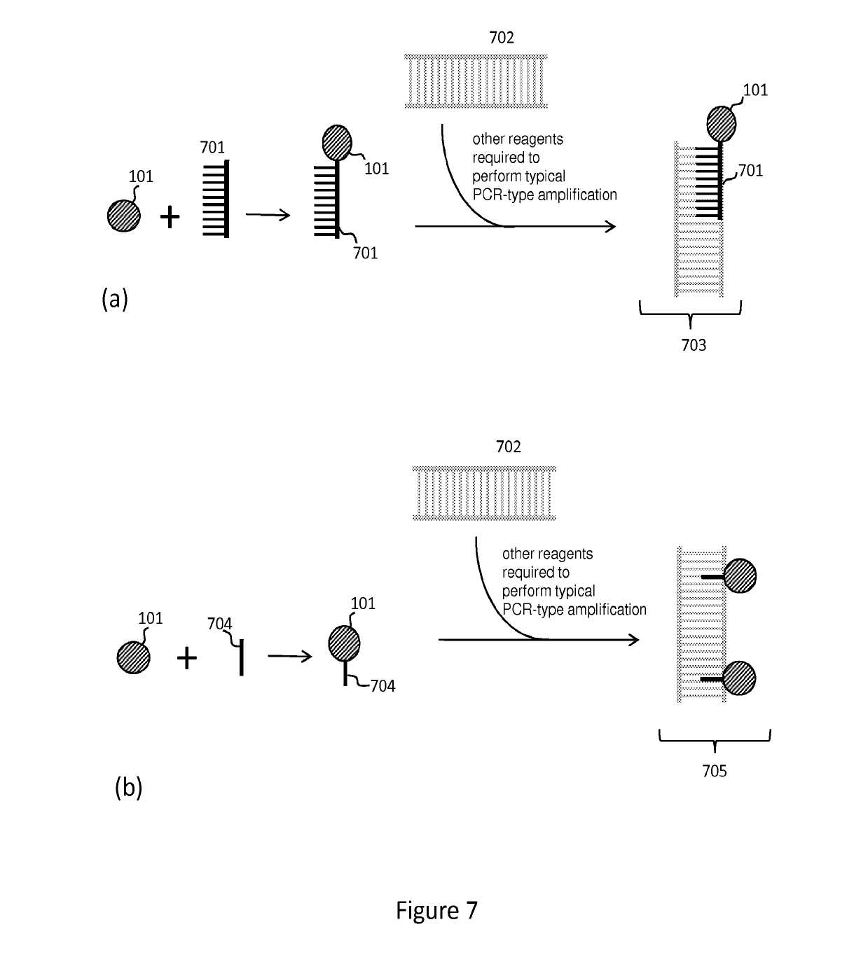

In yet another embodiment, the method may comprise an alternate means of attaching a NI to a target. A target may be labeled with a NI during a target amplification step such as polymerase chain reaction (PCR). In one mode of this embodiment schematically illustrated in FIG. 7a, NI 101 is first coupled to nucleic acid primer 701 forming NI-labeled nucleic acid primer 101-701. NI-labeled nucleic acid primer 101-701 may be incorporated into nucleic acid target 702 during a PCR-type amplification reaction, creating NI-labeled target 703. In an alternate mode of this embodiment schematically illustrated in FIG. 7b, NI 101 may be coupled to deoxyribonucleotide triphosphate (dNTP) 704 forming NI-labeled dNTP 101-704. NI-labeled dNTP 101-704 may be incorporated into target 702 during a PCR-type amplification reaction forming NI-labeled target 705. NI-labeled targets 703 and 705 are functionally equivalent to NI-label-target complex 103-106 in FIG. 2a, and the same subsequent steps as described above in reference to FIG. 2a can thereby then be performed wherein probe element 107 is a nucleic acid probe.

In the steps illustrated in FIGS. 2c and 2d, the occurrence of this recognition event may be amplified to aid in detection of the pathogen. In FIG. 2c, NI label-target-probe complex 103-106-107 may be contacted with a polymerizable solution 201. NI label-target-probe complex 103-106-107 may then be exposed to light. Exposure of NI 101 to light may cause NI 101 to generate a reactive species that instigates a polymerization reaction in polymerizable solution 201. As shown in FIG. 2d, a polymer 201′ forms in the vicinity of NI label-target-probe complex 103-106-107 immobilized on substrate 108. The presence of polymer 201′ may be easily detected and indicates that a recognition event has occurred because polymer 201′ will only form where NI label 103 attached to target element 106 has been immobilized by way of complexing with probe element 107.

Each of the steps described above and illustrated in FIGS. 2a-2d may be conducted under conditions favorable to promote complexing and hybridization as known to one of ordinary skill in the art. In one aspect of the embodiments disclosed herein, the method is performed under ambient conditions. In other words, the methods may be carried out without the need to purge the environment of oxygen, at room temperature, and at 1 atm. The steps described above may be performed in the order in which they are discussed, i.e., sequentially.

Nanoparticle initiator 101 discussed above may generally include nanoparticles of semiconductors, metal oxides and reduced metals. Specific examples include, but are not limited to, CdSe, CdS, InAs, InP, ZnS, CdTe, doped Si, TiO₂, Fe₂O₃, ZnO, Al₂O₃, Ag, Au, Co, Cu, Zn, Al, Ni, and Fe. In one aspect of the embodiments disclosed herein, the nanoparticle initiators may be quantum dots or QDots, which are nanometer-sized semiconductor particles having extraordinary size-tunable optical properties. QDots contain on the order of a few hundred to a few thousand atoms, and therefore have broad absorption spectra with extremely high cross-sections relative to most dye molecules. QDots may serve as nanoparticle initiators because they exhibit such high absorption cross-sections, have excellent long-term photochemical stability, and are known to generate electron/hole pairs as a result of photo-excitation.

The complexing between the various elements described above is not limited and may be any type of interaction binding one element to another. For example, the binding between elements may be based on bio-interactions, chemical interactions, or physical interactions. A bio-interaction may include, but is not limited to, antibody-antigen binding, enzyme-receptor binding, and nucleic acid hybridization. A chemical interaction may include, but is not limited to, interactions between small molecules, atoms, ions, surface charges, or any other molecular entity not considered to be macromolecular. A physical interaction may include, but is not limited to, an interaction based on shape, temperature, color, electrical conductivity, thermal conductivity, boiling point, melting point, refractive index, magnetism, photosensitivity, motion, pressure, viscosity, and other thermal, optical, electrical, magnetic, and mechanical properties known to those skilled in the art.

Recognition element 102, recognition elements 104 and 105 comprising target element 106, and probe element 107 discussed above may be any type of elements capable of binding together. The elements may include, but are not limited to, one or more of bio-recognition elements and chemical recognition elements. A bio-recognition element may include, but is not limited to, nucleic acids, proteins, antibodies, monoclonal antibodies and antisera reactive with specific antigenic determinants (such as on viruses, cells or other materials), peptides, aptamers, enzymes, enzyme substrates, substrate analogs, transition state analogs, cells, cellular membranes, organelles, cell membrane receptors, agonists and antagonists for cell membrane receptors, toxins and venoms, viral epitopes, hormones, hormone receptors, cofactors, drugs, polynucleotides, lectins, sugars, polysaccharides. A chemical recognition element may include, but is not limited to, small molecules, atoms, ions, surface charges, or any other molecular entity not considered to be macromolecular.

In one embodiment, biotin may be incorporated to target element 106 during a PCR-based amplification or may be conjugated to target element 106 using conjugation procedures known to those skilled in the art. Additionally, a NI may be conjugated to avidin or streptavidin using conjugation procedures known to those skilled in the art. The NI label incorporating avidin or streptavidin may be incubated with a biotinylated target-probe complex 106-107 to result in the formation of NI avidin label-biotinylated target-probe complex or NI strepdavidin-biotinylated target-probe complex 103-106-107. In this embodiment, the NI label-target-probe complex 103-106-107 may be immobilized on substrate 108.

For the purposes of the present invention, the term “nucleic acid” includes both single and double-stranded DNA and RNA. The term is also intended to include a strand that is a mixture of nucleic acids and nucleic acid analogs and/or nucleotide analogs, or that is made entirely of nucleic acid analogs and/or nucleotide analogs and that may be conjugated to a linker molecule. A “nucleic acid analog” is a modified nucleic acid or species unrelated to a nucleic acid capable of selectively binding to nucleic acids or other nucleic acid analogs. As used herein, the term “nucleotide analogs” includes nucleic acids where the phosphate backbone, internucleotide phosphodiester bond, sugar moieties, or nucleobases are modified to enhance bio-stability and “tune” the selectivity/specificity for target molecules.

In one aspect of the embodiments disclosed herein, the recognition event between the target and the probe may be sufficiently selective so as to avoid cross-hybridizing events. That is to say, probe element 107 may only bind with one type of target element 106 (e.g., a specific pathogen). The selectivity of the recognition event may be tuned by changing a variety of variables, including but not limited to temperature, ionic strength, pH, density of immobilization, length of the nucleic acid, substrate surface chemistry, and presence of crowding agents that limit non-specific interactions, blocking agents, and/or detergents.

In one embodiment disclosed herein, the method may comprise a step of substantially removing any NI label-target complex 103-106 that does not form NI label-target-probe complex 103-106-107 with probe element 107. Such a step may be performed prior to contacting NI label-target-probe element 103-106-107 with polymerizable solution 201. Such a step may be performed to ensure that reactive species that may instigate polymerization are not produced by exposing to light NI label-target complexes 103-106 that have not undergone a recognition event with probe element 107 (and would therefore result in amplification of a false positive).

Upon formation NI label-target-probe complex 103-106-107, NI label-target-probe complex 103-106-107 may be contacted with polymerizable solution 201. Polymerizable solution 201 may be any solution that polymerizes upon releasing a reactive species into polymerizable solution 201. In one embodiment, polymerizable solution 201 may be comprised of a polymer precursor. As used herein, polymer precursor is defined as a molecule or portion thereof which can be polymerized to form a polymer or copolymer. Polymer precursors may include any substance that contains an unsaturated moiety or other functionality that can be used in chain polymerization, or other moiety that may be polymerized in other ways. Such precursors include monomers and oligomers. One specific example of a polymer precursor suitable for use in the methods disclosed herein is poly(ethylene glycol diacrylate). PEGDA 575 is a form of poly(ethylene glycol diacrylate) that is commercially available from Sigma Aldrich. Other lengths/molecular weights of PEGDA may also be used.

Polymerizable solution 201 may further comprise a co-iniator or a co-catalyst. The co-catalyst or co-initiator may be triethanolamine, and is believed to function as an electron scavenger and a propagator of free radicals. Other species that may function as the co-catalyst or co-initiator include, but are not limited to, triethylamine, ethanolamine, N-methyl diethanolamine, N,N-dimethyl benzylamine, dibenzyl amine, N-benzyl ethanolamine, N-isopropyl benzylamine, tetramethyl ethylenediamine, lysine, ornithine, histidine, arginine, and other such molecules.

Polymerizable solution 201 may further comprise an inhibitor. An inhibitor can be added to polymerizable solution 201 to limit bulk polymerization that can occur as a result of impurities and/or trace free radicals formed by absorption of the irradiating light by species other than the intended nanoparticle initiator. Inhibitors may include, but are not limited to, hydroquinone, parabenzoquinone, tetrachloro-p-benzoquinone, ascorbic acid (vitamin C), retinol (vitamin A), alpha-tocopherol (vitamin E), butylated hydroxyanisole (BHA), butylated hydroxytoluene (BHT), furan derivatives such as dimethylfuran (DMF) and diphenylfuran (DPF), anthracene derivatives such as anthroquinone, and oxygen.

After NI label-target-probe complex 103-106-107 is contacted with polymerizable solution, NI label-target-probe complex 103-106-107 may be exposed to light in order to initiate polymerization. The light may be any type of light that will cause reactive species to leave NI 101 and initiate polymerization. The light used to generate a reactive species from NI 101 may have a specific wavelength known to one of ordinary skill in the art to cause NI 101 to generate reactive species. In one aspect of the embodiments disclosed herein, NI 101 is not consumed upon being exposed to light. In other words, NI 101 is capable of producing additional reactive species upon further exposure to light.

The method described above may comprise a further step of detecting polymer 201′ formed in the vicinity of NI label-target-probe complex 103-106-107. Such detection may confirm the presence of a pathogen. The method of detecting polymer 201′ formed is not limited. In one embodiment, the formed polymer 201′ may be present in high enough quantity to be visible to the naked eye, or without instrumentation. Polymer 201′ may also be visually detected using instrumentation, such as a microscope. In another embodiment, polymer 201′ formed can be subsequently rendered colored (e.g., red), thus making polymer 201′ more visible. Coloring polymer 201′ may be achieved by, e.g., contacting it with a solution of a simple chromophore for subsequent visualization. In yet another embodiment, the formed polymer 201′ and subsequently rendered colored can be visualized using an inexpensive transmission-based instrument capable of capturing an image of the resulting polymer spots or areas.

Other polymer precursors that enhance detection may include a colored (chromophoric) polymer, a fluorescent polymer, a magnetic polymer, or an electrically conductive polymer. Chromophoric polymers may be detected visually, with or without instrumentation. Electrically conductive polymers and magnetic polymers may be detected using any instrumentation capable of detecting magnetism and electrical conductivity.

In another embodiment, the method may be used to detect more than one target pathogen at the same time. In such a method, two or more different types of targets may be complexed with an NI label. For example, the method may utilize a plurality of a target pathogen A and a plurality of a target pathogen B. Correspondingly, two or more different types of probes may be made available to form complexes with the NI-label-target complexes, wherein each type of probe only forms a complex with one type of target pathogen.

Each of the probes may be immobilized on a substrate. Additionally, the location or spatial registry as well as the type of probe may be recorded. For example, the probes may be organized on a substrate in a 5 by 5 grid such that the first, third and fifth columns are known to be probes that complex with only target pathogen A, while the probes in the second and fourth columns are known to be probes that complex with only target pathogen B.

After contacting the different types of targets (as part of a NI label-target complex) with the different types of probes, the NI label-target-probe complexes may be contacted with polymerizable solution and exposed to light to initiate polymerization as discussed in greater detail above. Detection methods, including those described in greater detail above, may then be used to determine where polymer has formed and correspondingly where a recognition event between a target and probe has occurred. Furthermore, the location of the formed polymer on the substrate may be used to identify the presence and type of the various pathogens. Using the same example as described above, the formation of polymer in the first, third or fifth column confirms the presence of target pathogen A, while the formation of polymer in the second or fourth column confirms the presence of target pathogen B. If no polymer is formed in the second or fourth column, this confirms that the tested sample of targets did not contain target pathogen B.

As used herein, “comprising” is synonymous with “including,” “containing,” or “characterized by,” and is inclusive or open-ended and does not exclude additional, unrecited elements or method steps. As used herein, “consisting of” excludes any element, step, or ingredient not specified in the claim element. As used herein, “consisting essentially of” does not exclude materials or steps that do not materially affect the basic and novel characteristics of the claim. Any recitation herein of the term “comprising”, particularly in a description of components of a composition or in a description of elements of a device, is understood to encompass those compositions and methods consisting essentially of and consisting of the recited components or elements. The invention illustratively described herein suitably may be practiced in the absence of any element or elements, limitation or limitations which is not specifically disclosed herein.

These and other features and advantages of the disclosed method reside in the construction of parts and the combination thereof, the mode of operation and use, as will become more apparent from the following description, reference being made to the accompanying drawings that form a part of this specification wherein like reference characters designate corresponding parts in the several views. The embodiments and features thereof are described and illustrated in conjunction with systems, tools and methods which are meant to exemplify and to illustrate, not being limiting in scope.

In general the terms and phrases used herein have their art-recognized meaning, which can be found by reference to standard texts, journal references and contexts known to those skilled in the art. The above definitions are provided to clarify their specific use in the context of the invention.

All patents and publications mentioned in the specification are indicative of the levels of skill of those skilled in the art to which the invention pertains.

All references cited herein are hereby incorporated by reference to the extent that there is no inconsistency with the disclosure of this specification. Some references provided herein are incorporated by reference herein to provide details concerning additional starting materials, additional methods of synthesis, additional methods of analysis and uses of the invention.

### EXAMPLES

### Example 1

### Biotin-Streptavidin-QDot 545

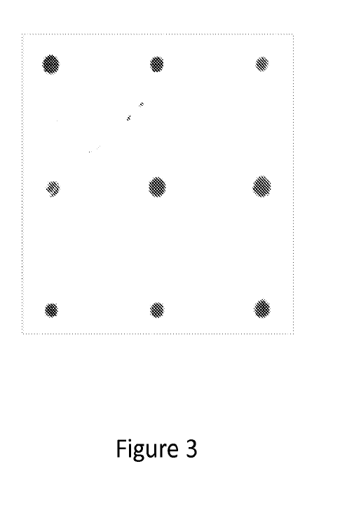

Proof-of-principle was demonstrated by hybridizing streptavidin-conjugated quantum dots (QDot 545, Invitrogen Corp., Carlsbad, Calif.) and biotinylated oligos to a surface-immobilized oligonucleotide, as outlined in FIG. 2b. Biotin-streptavidin is one of the strongest biological non-covalent interactions known, with a dissociation constant of ˜1×10⁻¹⁴. For this reason, it is used widely in a range of molecular, immunological, and cellular assays. Biotin as a primer tag provides a general labeling of all PCR products for detection, thereby, reducing label costs. Specifically, an oligonucleotide with a 5′-amino terminus and 3′-biotin terminus was spotted onto an aldehyde coated microarray glass in a 3×3 array (the amino terminus binds via Schiff base chemistry, such that the biotin terminus is directed toward solution). The streptavidin conjugated QDot 545 label, at 10 nM, was incubated with the microarray for one hour in a humidity controlled environment. The array was washed on an orbital shaker using the following protocol: 0.1% SDS (in 2×SSC) for one minute, 2×SSC for five minutes, and 0.2×SSC for five minutes. A volume of 40 μL of the monomer reagent mixture was added to the array, and irradiated with 405 nm light pulsed at 32 Hz for ˜90 seconds. The QDot 545 extinction coefficient at 405 nm is 730,000 M⁻¹ cm⁻¹. The monomer reagent mixture contained poly(ethyleneglycol diacrylate) (PEGDA, 575), water, and triethanolamine. After irradiation, excess monomer mixture was rinsed from the array and any resulting polymer stained with a water:methanol solution of Congo Red for 2 minutes. A representative image of this model microarray system is shown in FIG. 3, where each spot contains less than a monolayer of biotinylated oligos. All images presented herein were acquired with an inexpensive digital camera-based transmission instrument (specifically, for these studies InDevR's IntelliChip Reader was used although any commercial instrument based on reflectance or transmission would produce sufficient images) and darker spots indicate presence of stained polymer.

### Example 2

### Detection of Group A Streptococcus (GAS) Bacteria

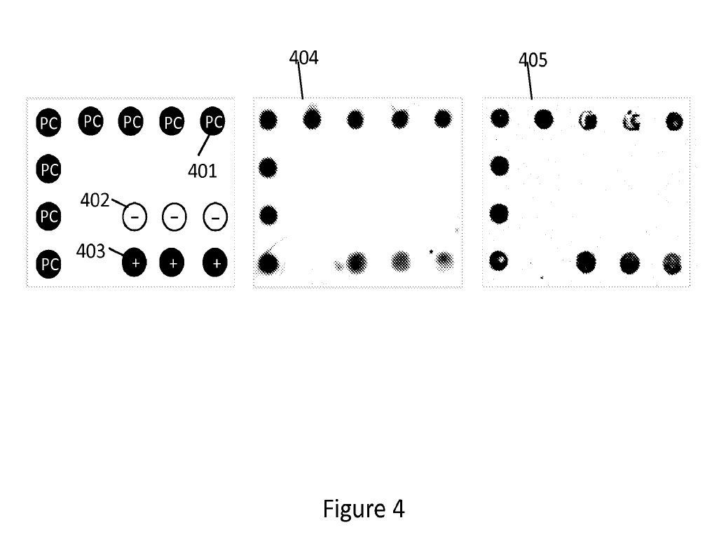

The microarray layout for detection of Streptococcus pyogenes (Group A Streptococcus, GAS) is shown in FIG. 4, where an immobilized probe sequence complementary to a biotinylated target oligo was used as the positive control and spatial marker 401, the GAS immobilized probe sequence 403 was spotted in triplicate, and an immobilized probe sequence 402 specific for influenza A also spotted in triplicate was used as a negative control. S. pyogenes (GAS) genomic DNA was obtained from American Type Culture Collection (ATCC, Manassas, Va., #700294D-5). Genomic DNA was amplified by PCR with the primer incorporating biotin and the opposite strand enzymatically digested to form ssDNA (i.e., the target) for hybridization to the microarray. Hybridization was conducted for one hour in a humidity controlled environment at room temperature. Excess target was removed with 2×SSC on an orbital shaker for 1 minute. The labeling and polymerization protocols described above were used for signal enhancement. In FIG. 4, representative image 404 demonstrates proof-of-principle for detection of a genomic material from Group A Streptococcus.

PCR conditions were as follows: PCR reactions were performed with 2 μL of template DNA using the Tfi polymerase kit (Invitrogen Corporation, Carlsbad, Calif.) with final concentrations of 2 U/rxn Tfi polymerase, 1× reaction buffer, 1.5 mM MgCl₂, 0.2 mM in each dNTP, 0.6 μM in each primer in a total 20 μL reaction volume. An initial melt at 94° C. for 30s was performed, followed by 45 cycles of the following: 94° C. for 30s, 55° C. for 30s, 72° C. for 1 min. A final extension at 72° C. for 7 min was then performed and samples were held at 4° C. until further analysis. Gel electrophoresis was then performed on 4 μL of PCR product using a pre-cast, ethidium bromide stained 4% agarose EGel (Invitrogen Corporation, Carlsbad, Calif.). The gel was allowed to run for 30 min, and subsequently visualized using the Alpha DigiDoc RT2 gel documentation system (Alpha Innotech Corporation, San Leandro, Calif.).

### Example 3

### Detection of Group A Streptococcus (GAS) Bacteria from a Clinical Sample

Patient samples in the form of throat swabs were obtained from the Boulder Community Hospital Microbiology Laboratory (Boulder, Colo.). Swabs were stored in a tube containing a sponge soaked in Liquid Stuart Medium, and kept refrigerated at 4° C. until extracted. Nucleic acid was extracted from swabs using the Bacterial Xpress™ nucleic acid extraction kit (Millipore, Billerica, Mass.) according to the manufacturer's instructions. PCR, enzymatic digestion, hybridization to the microarray, and signal amplification were conducted as described previously. In FIG. 4, representative image 405 resulted from detection of a fragment of a single gene from Streptococcus pyogenes after extraction from a patient sample and PCR amplification.

### Example 4

### Quantitative Comparison with Fluorescence Detection

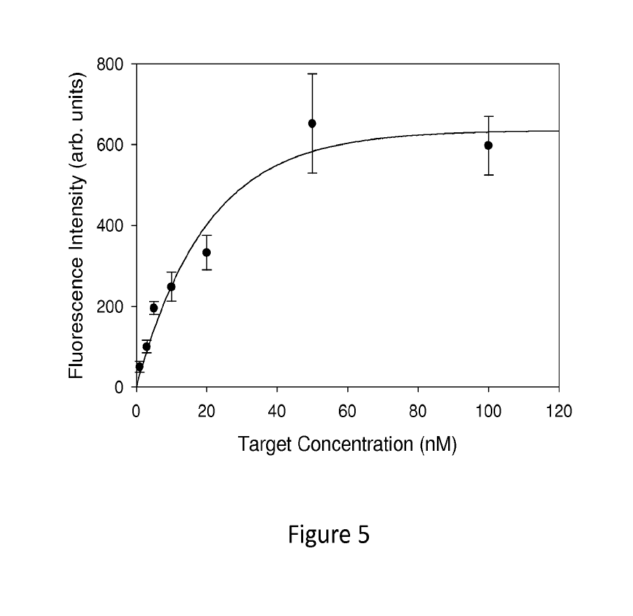

In order to evaluate the detection sensitivity of nanoparticle initiated polymerization relative to standard fluorescence detection, a direct comparison was conducted on a low density microarray. The microarray layout was identical to that shown in FIG. 4. In this case, hybridization of the positive control capture sequence was conducted with a biotinylated target (i.e., the configuration shown in FIG. 2b). After incubation with the QDot 565/streptavidin conjugate, the array was imaged in a Genetix aQuire Confocal fluorescence microarray scanner with 532 nm excitation, 570 nm emission, and a 60% detector setting. All reagents were held constant while the concentration of the biotinylated target was varied from 1 nM to 100 nM, and this data is shown in FIG. 5. For the particular experimental conditions used in this study, both fluorescence and nanoparticle initiated polymerization were detected at the lowest target concentration (1 nM). The lowest detected concentration is near the calculated limit of detection (based on a signal three times the background) for fluorescence. This study indicates that nanoparticle initiated polymerization is competitive with fluorescence detection in terms of sensitivity.

### Example 5

### Silver Nanoparticle Initiator

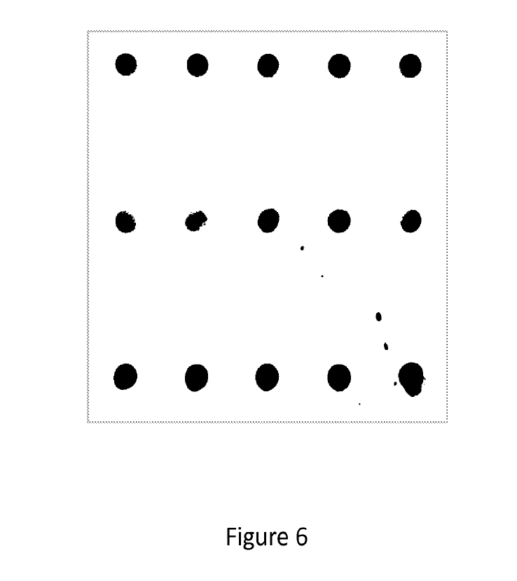

Ag nanoparticles (<100 nm diameter) were used to demonstrate proof-of-principle with a second type of nanoparticle initiator, in this case a reduced metal rather than a mixed metal (e.g., CdSe QDot). Ag nanoparticles were spotted onto aldehyde-coated glass slides in a layout consisting of 5 spots per row and 3 rows. After drying, excess Ag nanoparticles were rinsed from the surface until no particles could be detected visually. The monomer reagent mixture was added and polymerization conducted as described previously. Thick polymer film formed only where Ag had been spotted. A resulting image of the polymer spots after staining with Congo Red solution is shown in FIG. 6. This study demonstrated the utility of Ag nanoparticles for initiation of polymerization for signal enhancement and also served to demonstrate that a broad range of nanoparticle types can be used to initiate polymerization after a biological, chemical, or physical recognition event. It is anticipated that several types of nanoparticles can be used to initiate polymerization upon excitation with light, including but not limited to semiconductors (e.g., CdSe, doped Si, TiO₂, CdS, InAs, InP, ZnS, CdTe), some metal oxides (e.g., Fe₂O₃, ZnO, Al₂O₃), and some reduced metals (e.g., Ag, Au, Co, Cu, Zn, Al, Ni, Fe).

What is claimed is:

1. A method for amplifying a recognition event between a target and a probe comprising the steps of:
(a) conjugating a nanoparticle initiator to a recognition element to form a nanoparticle initiator label;
(b) contacting the nanoparticle initiator label with a target element to form a nanoparticle initiator label-target complex;
(c) contacting the nanoparticle initiator label-target complex with a probe element to form a nanoparticle initiator label-target-probe complex;
(d) contacting the nanoparticle initiator label-target-probe complex with a polymerizable solution comprising a polymer precursor; and
(e) exposing the nanoparticle initiator label-target-probe complex and the polymerizable solution to light to form a polymer.

2. The method as claimed in claim 1 wherein steps (a) through (e) are performed sequentially.

3. The method as claimed in claim 1, further comprising:
(f) detecting the polymer formed in step (e), thereby detecting an amplified target-probe interaction.

4. The method as claimed in claim 3, wherein detecting the polymer comprises visual detection with instrumentation, visual detection without instrumentation, magnetic detection, or electrical conductivity detection.

5. The method as claimed in claim 1, wherein the nanoparticle initiator comprises CdSe, doped Si, TiO₂, CdS, InAs, InP, ZnS, CdTe, Fe₂O₃, ZnO, Al₂O₃, Ag, Au, Co, Cu, Zn, Al, Ni, or Fe.

6. The method as claimed in claim 1, further comprising a step of substantially removing any nanoparticle initiator label-target complex that does not form a nanoparticle intiator label-target-probe complex with the probe element between steps (c) and (d).

7. The method as claimed in claim 1, wherein the nanoparticle initiator is not consumed upon being exposed to light.

8. The method as claimed in claim 1, wherein the method is carried out under ambient conditions.

9. The method as claimed in claim 1, wherein the polymerizable solution further comprises a co-initiator or a co-catalyst.

10. The method as claimed in claim 1, wherein the polymerizable solution further comprises an inhibitor.

11. The method as claimed in claim 1, wherein the polymer formed comprises a chromophoric, fluorescent, magnetic, or electrically conductive polymer.

12. The method of claim 1, wherein the conjugation of the nanoparticle initiator to the target element takes place during PCR-based amplification of the target element.

13. The method as claimed in claim 1, wherein:
the target element comprises a plurality of two or more different types of target elements;
the probe element comprises a plurality of two or more different types of probe elements, each of the plurality of two or more different types of probe elements immobilized at a location on a substrate and designed to form complexes with one of the two or more different types of target elements; and
wherein the location on the substrate and the type of target element to which each of the plurality of the two or more different types of probe elements is known, such that a target element may be identified based on the location on the substrate where the polymer forms.

14. A method for amplifying a recognition event between a target and a probe comprising the steps of:
(a) contacting a target element with a probe element to form a target-probe complex;
(b) conjugating a nanoparticle initiator to a recognition element to form a nanoparticle initiator label;
(c) contacting the target-probe complex with the nanoparticle initiator label to form a nanoparticle initiator label-target-probe complex;
(d) contacting the nanoparticle initiator label-target-probe complex with a polymerizable solution comprising a polymer precursor; and
(e) exposing the nanoparticle initiator label-target-probe complex and the polymerizable solution to light to form a polymer.

15. The method as claimed in claim 14 wherein steps (a) through (e) are performed sequentially.

16. The method as claimed in claim 14, further comprising:
(f) detecting the polymer formed in step (e), thereby detecting an amplified target-probe interaction.

17. The method as claimed in claim 16, wherein detecting the polymer comprises visual detection with instrumentation, visual detection without instrumentation, magnetic detection, or electrical conductivity detection.

18. The method as claimed in claim 14, wherein the nanoparticle initiator comprises CdSe, doped Si, TiO₂, CdS, InAs, InP, ZnS, CdTe, Fe₂O₃, ZnO, Al₂O₃, Ag, Au, Co, Cu, Zn, Al, Ni, or Fe.

19. The method as claimed in claim 14, further comprising a step of substantially removing any target element that does not form a target-probe complex with the probe element prior to step (c).

20. The method as claimed in claim 14, wherein the nanoparticle initiator is not consumed upon being exposed to light.

21. The method as claimed in claim 14, wherein the method is carried out under ambient conditions.

22. The method as claimed in claim 14, wherein the polymerizable solution further comprises a co-initiator or a co-catalyst.

23. The method as claimed in claim 14, wherein the polymerizable solution further comprises an inhibitor.

24. The method as claimed in claim 14, wherein the polymer formed comprises a chromophoric, fluorescent, magnetic, or electrically conductive polymer.

25. The method as claimed in claim 13, wherein:
the target element comprises a plurality of two or more different types of target elements;
the probe element comprises a plurality of two or more different types of probe elements, each of the plurality of two or more different types of probe elements immobilized at a location on a substrate and designed to form complexes with one of the two or more different types of target elements; and
wherein the location on the substrate and the type of target element to which each of the plurality of the two or more different types of probe elements is known, such that a target element may be identified based on the location on the substrate where the polymer forms.
